(19) United States
(12) Patent Application Publication
(10) Pub. No.: US 2003/0229913 A1
(43) Pub. Date: Dec. 11, 2003

(54) Identification of the flt1 gene required for angiogenesis in zebrafish and uses thereof

(76) Inventors: Hinrich Alexander Habeck, Tuebingen (DE); Stefan Schulte-Merker, Tuebingen (DE)

Correspondence Address: JAN P. BRUNELLE, EXELIXIS, INC., 170 HARBOR WAY, P.O. BOX 511, SOUTH SAN FRANCISCO, CA, 94083-0511, US

(21) Appl. No.: 10/402,365

(22) Filed: Mar. 27, 2003

Related U.S. Application Data
(60) Provisional application No. 60/368,616, filed on Mar. 27, 2002.

(51) Int. Cl. A01K 67/027; C12Q 1/68

(52) U.S. Cl. 800/20; 435/6

(57) ABSTRACT

An engineered mutant teleost embryo having reduced flt1 activity that causes a phenotype of normal assembly of main circulatory routes and a reduction in sprouted blood vessels is described. Methods of using the mutant teleost embryo for identifying genes that interact with flt1 are also described.

### REFERENCE TO RELATED APPLICATIONS

This application claims priority to U.S. provisional patent application 60/368,616 filed Mar. 27, 2002. The content of the prior application is hereby incorporated in its entirety.

### FIELD OF THE INVENTION

The invention relates to the identification of flt1 gene function in zebrafish, which is required for sprouting angiogenesis, and uses of flt1 mutant zebrafish in genetic and compound screens to identify members of the flt1 signaling pathway and compounds that affect sprouting angiogenesis.

### BACKROUND OF THE INVENTION

The formation of vasculature is an essential process during embryonic development. Two different mechanisms for the generation of blood vessels have been distinguished: vasculogenesis and angiogenesis (Risau W, Flamme I., Annu Rev Cell Dev Biol (1995) 11:73-91; Risau W., Nature (1997) 386:671-674; and Carmeliet P., Nat Med (2000) 6:389-395). In the course of vasculogenesis, the first vessels are formed by the assembly of mesodermal precursors (angioblasts) and by their subsequent differentiation into endothelial cells. Once a primitive network of vascular tubes has been established additional vessels grow by sprouting from the preexisting vasculature. This process is called angiogenesis.

The molecular mechanisms of vasculogenesis and angiogenesis have been studied intensively, especially because the formation of new blood vessels plays an important role in a variety of diseases (Folkman J, Nat Med (1995) 1:27-31; and Ferrara N, Alitalo K, Nat Med (1999) 5:1359-1364). Gene targeting experiments in mouse have shown that the vascular endothelial growth factor (VEGF) and its high affinity receptors Kdr (Flk1, VEGFR2) and Flt1 (VEGFR1) are crucial for the early steps of vessel formation. The loss of VEGF or Kdr function leads to a severe reduction of hematopoietic precursors and impairs vasculogenesis (Carmeliet P, et al., Nature (1996) 380:435-439; Ferrara N., et al., Nature (1996) 380:439-442; and Shalaby F. et al., Nature (1995) 376:62-66). In contrast, flt1 null mutants display an increased number of hematopoietic and endothelial precursors and blood vessels form, but are disorganized (Fong G H et al., Nature (1995) 376:66-70; and Fong G H et al., Development (1999) 126:3015-3025.). Surprisingly, the deletion of the tyrosine kinase domain of flt1 has no effect on the development of the vasculature (Hiratsuka et al., Proc Natl Acad Sci USA (1998) 95:9349-9354). These experiments suggest, that Kdr acts as a positive regulator of endothelial specification and proliferation, whereas Flt1 appears to be a negative regulator.

In zebrafish, a partial sequence for one VEGF receptor has been described (Fouquet B, et al., Dev Biol (1997) 183:37-48; Liao et al., Development (1997) 124:381-389; Sumoy L, et al., Mech Dev (1997) 63:15-27; and Thompson M A, et al., Dev Biol (1998) 197: 248-269). A comparison of this partial sequence with Flk1 and Flt1 from mouse and human showed that it was more closely related to Flk1 (Kdr, Vegfr2). Investigators were unable to identify a second VEGF receptor in zebrafish; thus, it was presumed that the zebrafish flk1 ortholog represents an ancestral gene that gave rise to mammalian flk1 and Flt1 (Liao et al., supra). Partial and complete Flk1 nucleic acid and protein sequences from zebrafish are published in GenBank (Genbank Identifier (GI) numbers: 6066286, 6066287, 18031944, 18031945, 1785865, 1785866, 1661231, and 1661232).

Vasculogenesis in zebrafish has been studied by analyzing the expression pattern of the cloned VEGF receptor and other genes expressed in angioblasts (Liao E C, et al., Genes Dev (1998) 12:621-626; Gering M, et al., Embo J (1998) 17: 4029-4045; and Brown L A, et al., Mech Dev (2000) 90:237-252). At 12 hours post fertilization (hpf) angioblasts are found as two pairs of bilateral lateral stripes, one in the lateral plate mesoderm of the trunk region, the other one in the head region. These stripes expand rostrally and caudally as the embryos develop. The endothelial precursors in the head break up into two plexus outlining the primordia of the head vessels. At 16 hpf the angioblasts in the mid-trunk region start to converge towards the midline. This convergence extends caudally. The angioblasts merge into a single stripe directly underneath the notochord and differentiate into the dorsal aorta and posterior cardinal vein. At 30 hpf the vessels have formed a lumen and circulation starts. The further development of the vasculature has been studied in great detail employing the confocal microangiography technique (Weinstein B M, et al., Nat Med (1995) 1:1143-1147; and Isogai S, et al., Dev Biol (2001) 230:278-301.). The cloning of zebrafish vegf and the analysis of its expression pattern has shown that the ligand is expressed in close spatial and temporal proximity to its receptor (Liang D, et al., Biochim Biophys Acta (1998) 1397:14-20.).

The zebrafish is an ideal model organism to study the processes underlying vascular development taking advantage of the easy accessibility of the vasculature and the different possibilities of genetic manipulations. In forward genetic screens several mutations affecting the proper development of blood vessels have been identified (Weinstein B M, et al., supra; Stainier D Y, et al., Development (1995) 121:3141-3150; Stainier et al., Development (1996) 123:285-292; and Chen J N, et al., Development (1996) 123:293-302. The analysis and molecular cloning of some of these mutations especially improved the understanding of how vessels acquire their arterial or venous identities (Zhong T P, et al., Science (2000) 287:1820-1824; Zhong T P, et al., Nature (2001) 414:216-220; and Lawson N D, et al., Development (2001) 128:3675-3683. In a reverse genetics approach, it was shown that morpholino knock-down of zebrafish VEGF results in the failure of blood vessel formation (Nasevicius A, et al., Yeast (2000) 17:294-301). The overexpression of VEGF has recently been shown to stimulate not only endothelial cell differentiation but also hematopoiesis (Liang D, et al. Mech Dev (2001), 108: 29-43).

### SUMMARY OF THE INVENTION

The invention provides engineered mutant teleost embryos having reduced flt1 activity, which causes a phenotype of normal assembly of main circulatory routes and a reduction in sprouted blood vessels. The flt1 phenotype may be caused by an induced mutation of flt1, or may be caused by an exogenously added nucleic acid inhibitor that specifically inhibits flt1, such as an antisense phosphoramidate morpholino (PMO). In preferred embodiments, the teleost embryo is a zebrafish.

The mutant teleost embryos of the invention can be used in methods for identifying genes that interact with flt1. One method comprises crossing a teleost that is a heterozygous carrier of an induced mutation of flt1 with a second teleost that is a heterozygous carrier of an induced mutation in a gene of interest, and examining vasculature of progeny teleost embryos to determine whether the double heterozygous progeny displays changes in the vasculature. In another method, teleosts that are heterozygous carriers of an induced mutation of flt1 are crossed. Their eggs are contacted with a molecule that specifically inhibits a gene of interest. The eggs are cultured under conditions that allow formation of teleost embryos, and the vasculature of the teleost embryo is examined to determine whether the gene of interest modifies a flt1 phenotype of normal assembly of main circulatory routes and a reduction in sprouted blood vessels.

The invention also provides nucleic acid inhibitors that specifically inhibit flt1 gene function. In a preferred embodiment, the nucleic acid inhibitor is a PMO.

### BRIEF DESCRIPTION OF THE DRAWINGS

FIG. 1: In comparison to wildtype (FIG. 1a), Flt-mutant (FIG. 1b) zebrafish larvae at 4 dpf have defective vasculature. In particular, the intersegmental vessels (Se) reach to the dorsal side at only a few somite boundaries, while in the others they end at the horizontal myoseptum. In the mutant larvae, the parachordal vessel (PAV) is well-developed and connects the remaining Se. The scale bar is 200 μm.

FIG. 2: In Flt-mutants at 4 dpf, the subintestinal vein (SIV) is thin and interrupted and the number of branches is reduced (FIG. 2b) in comparison to wildtype larvae (FIG. 2a). The scale bar is 200 μm.

FIG. 3: Approximately 30% of the mutant larvae show an accumulation of erythrocytes either around the eyes (FIG. 3b), or above the yolk sac in the region of the developing swim bladder (FIG. 3d), in comparison to wildtype larvae (FIGS. 3a and 3c, respectively). The scale bar is 40 μm in FIGS. 3a and 3b, and 200 μm in FIGS. 3c, and 3d.

FIG. 4: Confocal microangiography of a wildtype (FIG. 4a) and a Flt mutant larva (FIG. 4b), lateral view, at 4 dpf shows that the main routes of the circulatory system are normally developed in mutant larvae. In the trunk and tail, the dorsal aorta (DA), caudal artery (CA), caudal vein (CV), and posterior cardinal vein (PCV) are indistinguishable between siblings and mutants. The mutant heart (H) is slightly smaller. At some somite boundaries the intersegmental vessels (Se) are lacking, while at the others they reach only the horizontal myoseptum and are connected by a parachordal vessel (PAV). In those somites where the Se reach the dorsal side a dorsal longitudinal anastomotic vessel (DLAV) forms. Only the most proximal parts of the subintestinal vein (SIV) can be detected while there is no continuous circulation across the yolk. Despite the lack of a functional SIV a network of small channels form in the developing liver (L). Other structures shown are aortic arches (AA), basilar artery (BA), dorsal longitudinal vessels (DLV), mesencephalic vein (MsV), posterior cerebral vein (PCeV), primary head sinus (PHS), vertebral artery (VTA). The scale bar is 200 μm.

FIG. 5: FIGS. 5a and 5b are dorsal views of the head of wildtype and Flt mutant larvae, respectively, as revealed by confocal microangiography at 4 dpf; and FIGS. 5c and 5d are schematic drawings of a subset of the head vessels. The vascular loop in the pectoral fin consisting of the pectoral artery (PA) and pectoral vein (PV) is not detectable in the mutant larvae. In the head, the vessels forming before 1.5 dpf, e.g. the prosencephalic artery (PrA), the anterior cerebral vein (ACeV), and the BA, are nonnally developed in the mutant. In the wild type, the central arteries (CtA) penetrate into the brain substance. In the mutant larvae these small caliber vessels are not detectable. The (*) in FIG. 5b indicates the accumulation of fluorescent dye around the right eye. Other structures depicted are basal communicating artery (BCA), cerebellar central artery (CCtA), DA, dorsal ciliary vein (DCV), middle cerebral vein (MCeV), middle mesencephalic central artery (MMCtA), metencephalic artery (MtA), nasal vein (NV), posterior cerebral vein (PCeV), PCV, primordial hindbrain channel (PHBC), primordial midbrain channel (PMBC), posterior mesencephalic central artery (PMCtA); and Se. The scale bar is 200 μm.

FIG. 6: FIGS. 6a and 6b are magnified views of vessels in a ventral layer. The section of the lateral dorsal aorta (LDA) between the confluence of the artery of the first branchial arch (AA3) and the caudal division of the internal carotid artery (CaDI) consists of a single vessel in the wild type (FIG. 6a). In the mutant, a vascular plexus forms instead (FIG. 6b). The ophthalmic vein (OpV) is also depicted. The scale bar is 20 μm.

FIG. 7: A phylogenetic tree depicts the relatedness of zebrafish Flt1 with VEGF receptors from other species, and demonstrates that the zebrafish gene previously published as kdr is the zebrafish orthologue of flt1. Genebank accession numbers of the sequences compared are Kdr (M. musculus): A46065; Kdr (R. norvegicus): NP_{—}037194; KDR (H. sapiens): AAC16450; QUEKI (C. coturnix): P52583; FLT4 (H. sapiens): P35916; Flt4 (M. musculus): P35917; QUEK2 (C. coturnix): CAA58267; Flt4 (D. rerio): AAD56011; Flt1 (M. musculus): NP_{—}034358; Flt1 (R. norvegicus): NP_{—}062179; FLT1 (H. sapiens): AAC16449; Flt1 (D. rerio): AAL16381.1.

### DETAILED DESCRIPTION OF THE INVENTION

In a large-scale forward genetic screen, we analyzed the circulatory system of zebrafish larvae at four days post fertilization (4 dpf). Rather than examining living embryos, as done in previous screens (Stainier D Y, et al., (1996) supra; and Chen et al., supra), we took advantage of the fact that endothelial cells possess a high endogenous alkaline phosphatase activity, and stained the vasculature of fixed embryos by adding a precipitating substrate. This improved both the accuracy of detecting vascular defects and the overall screen efficiency. At 4 dpf, normal development of the zebrafish vascular system includes both vasculogenic and angiogenic events. The main circulatory routes of the trunk and head are formed by the assembly of angioblasts and their differentiation into endothelial cells lining the blood vessels. Sprouting from preexisting vessels results in the formation of intersegmental vessels, blood supply of the digestive system, the central arteries of the brain, and the vessels of the pectoral fin. The earliest of these processes starts around 24 hours post fertilization, with the first intersegmental vessels beginning to branch from the dorsal aorta. But, it is not until 4 dpf that the pattern of the vasculature reaches a relatively stable status that allows the reliable identification of even subtle changes; hence, the selection of the time point in which to perform the screen.

In the screen we identified more than 700 mutations affecting the proper formation of blood vessels. In a detailed analysis of one of these mutants, “Schwentine”, we discovered that sprouting of blood vessels from preexisting vessels is specifically disrupted, while the assembly of the main circulatory routes (e.g. heart, dorsal aorta, caudal artery, etc.) occurs normally. Specifically, in Schwentine mutant embryos, the development of vessels is unaltered up to 1.5 dpf. After this time point, all vessels that normally form by sprouting from the existing vessels are reduced, absent or non-functional. We determined that the gene responsible for this mutant phenotype is the same gene that was previously published as kdr (Fouquet et al., supra). However, our analysis shows that this gene is actually the zebrafish orthologue of mammalian flt1 and that a mutation in this gene is responsible for the change in vascular development. Hence, we refer to the gene that was previously identified as zebrafish kdr as zebrafish flt1. The full-length DNA sequence for zebrafish flt1 is presented as SEQ ID NO:1, which includes previously unpublished 5′UTR (nucleotides 1-365). The amino acid sequence of the encoded Flt1 protein is presented in SEQ ID NO:2.

We further show that the expression of flt1 is downregulated in mutant embryos, while the expression of other markers for vasculogenesis and hematopoiesis is not changed in flt1 mutant embryos. Finally, we show that the overexpression of vegf₁₆₅ stimulates hematopoiesis in sibling and mutant embryos, and induces the sprouting of additional blood vessels in sibling, but never in flt1 mutant embryos. These results show that Flt1 activity is necessary for the formation or proper function of vessels originating from sprouting.

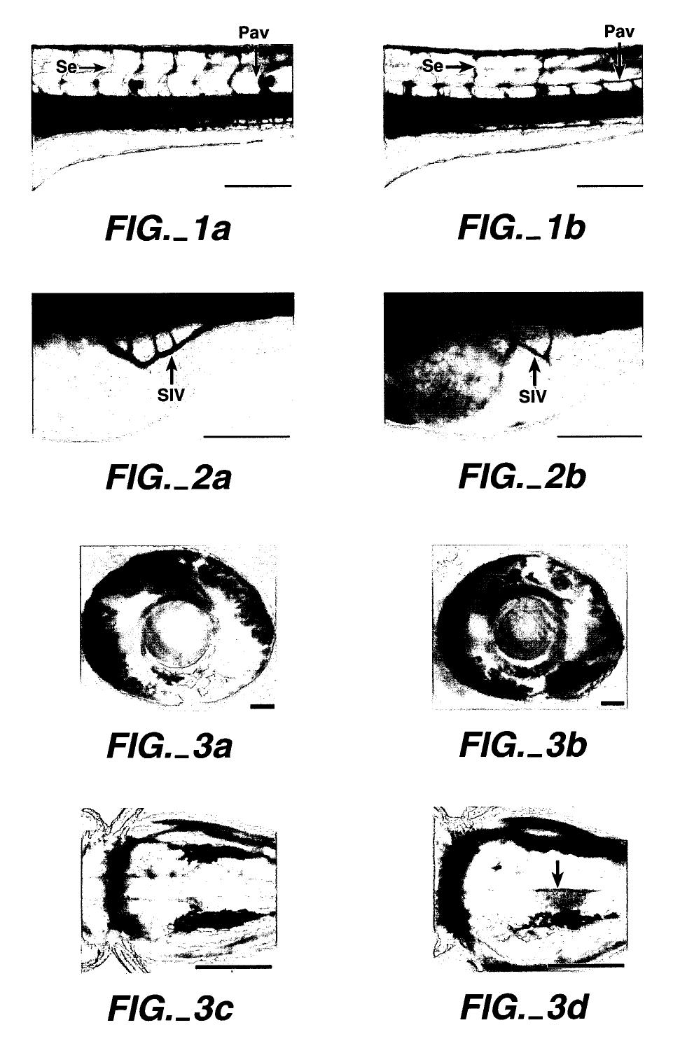

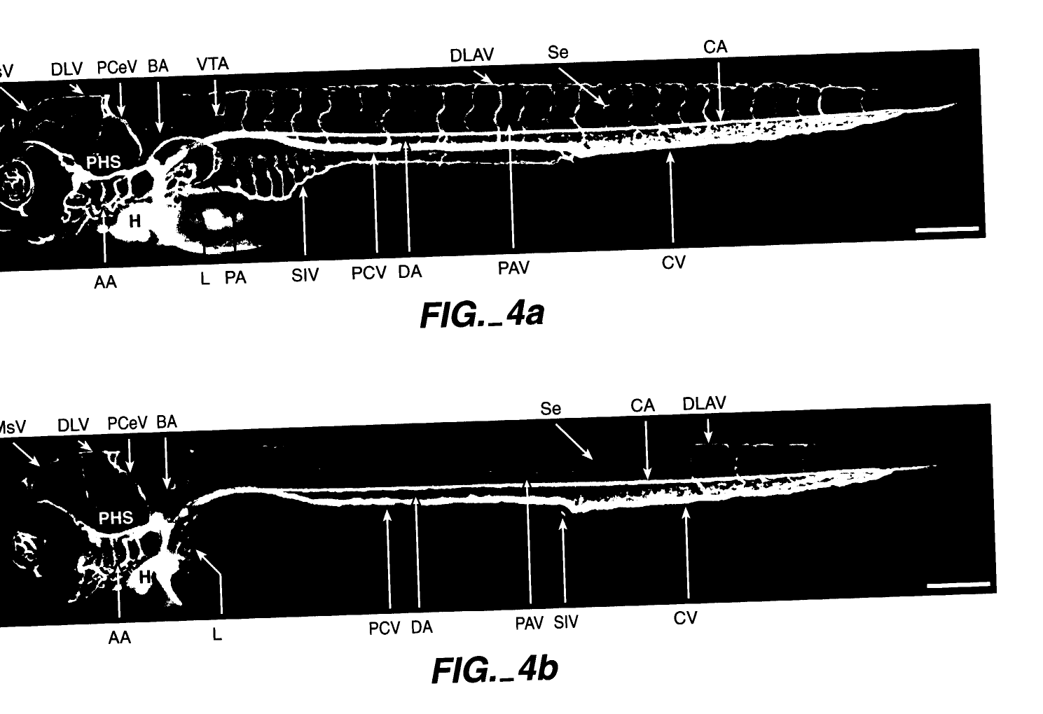

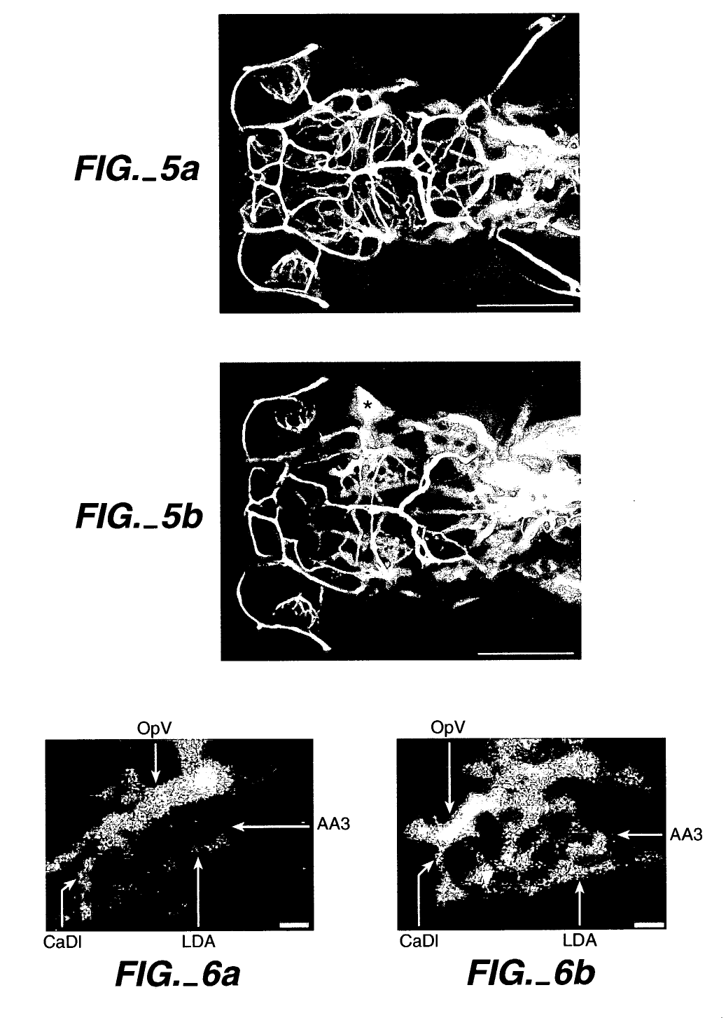

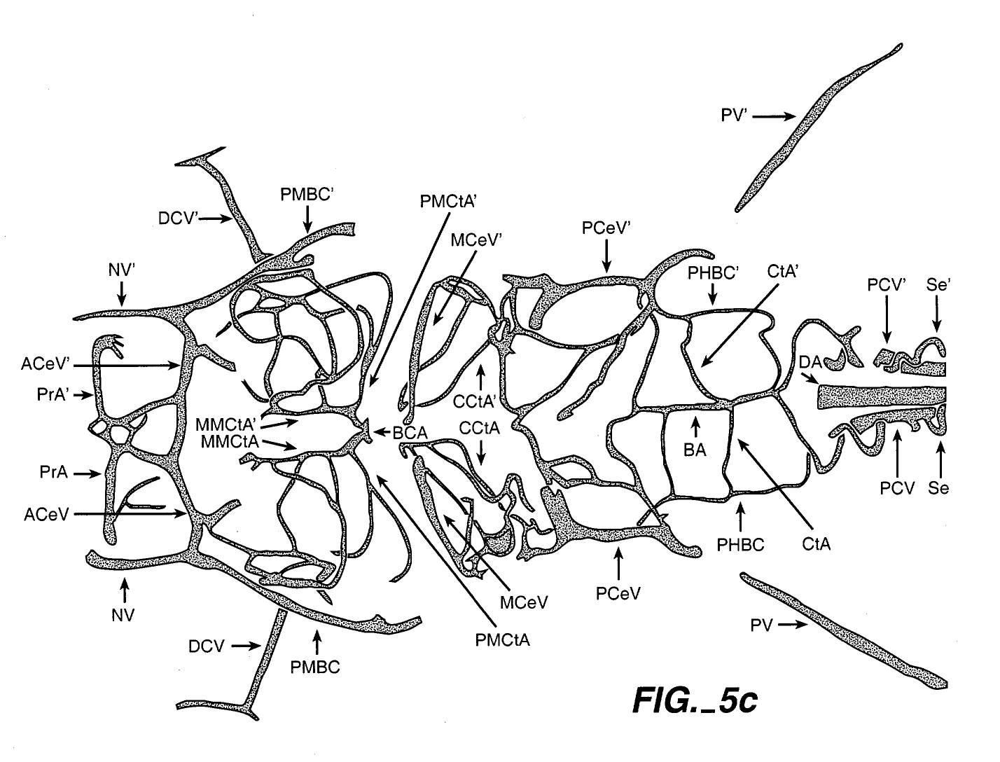

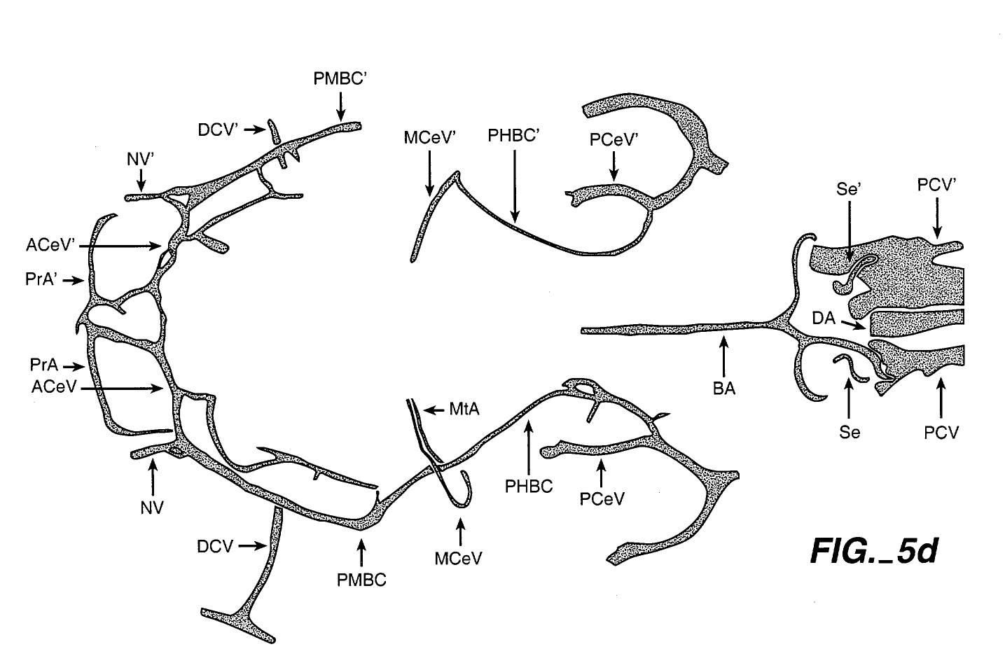

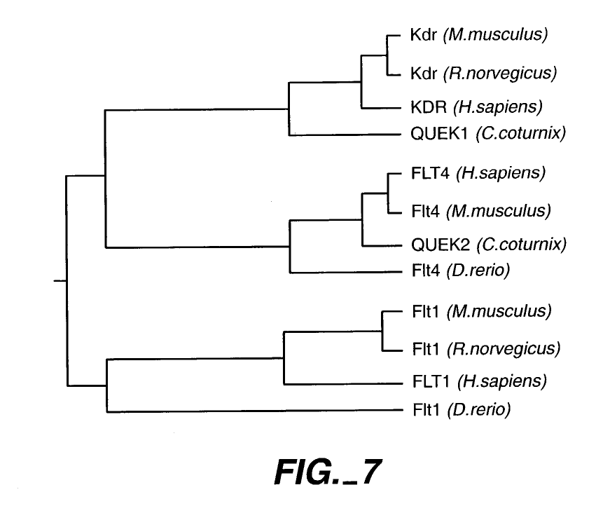

Accordingly, the invention provides engineered teleost embryos that have reduced FIt1 activity resulting in a flt1 phenotype. As used herein, “Flt1 activity” refers to the normal expression of the flt1 gene, including transcription and translation resulting in normal generation of the Flt1 protein. The term “flt1 phenotype” is used to describe a teleost embryo that has normal assembly of its main circulatory routes, but a reduction in sprouted blood vessels, relative to wild-type embryos. Specifically, the flt1 phenotype is characterized by a reduction of intersegmental vessels, as shown in FIG. 1b; a thin and interrupted subintestinal vein (SIV) with reduced branching, as shown in FIG. 2b; absence of the pectoral artery (PA) and pectoral vein (PV) vascular loop in the pectoral fin, as shown in FIG. 5b; and absence of all central arteries (CtA) in the head, as shown in FIG. 5b. The engineered teleost embryos can be used in genetic screens to identify interacting genes in the flt1 pathway, or in compound screens to identify pharmaceutical agents that promote angiogenesis. Preferred teleosts are zebrafish (Danio rerio) and medaka (Oryzias latipes).

Generation of Flt1 Mutant Teleosts

The term “engineered teleost” as used herein, means that the teleost is intentionally manipulated to cause a reduction in Flt1 activity, either by generating an induced mutation in the endogenous flt1 gene, or by inhibiting flt1 gene expression by nucleic acid interference. In one embodiment, the teleost may be engineered using methods known in the art for producing transgenic teleosts (see, e.g., Culp P et al., Proc Natl Acad Sci USA 1991, 88:7953-7957; Lin S, Methods Mol Biol 2000, 136:375-383; Koster R W and Fraser S E, Dev Biol 2001, 233:329-346; Hsiao C et al., Dev Dyn 2001, 220:323-326; Linney E et al., Dev Biol 1999, 213:207-216; Ju et al., Dev Genet 1999, 25:158-67; and Ma C et al., Proc Natl Acad Sci USA 2001, 98:2461-2466). Methods for homologous recombination are available in various non-human organisms and cells (e.g., Capecchi, Science 1989, 244:1288-1292; Joyner et al., Nature 1989, 338:153-156; Rong Y S and Golic K G, Science 2000, 288:2013-2018; Mateyak M K et al., Cell Growth Differ 1997, 8:1039-1048; and Frances V and Bastin M, Nucleic Acids Res 1996, 24:1999-2004). A “knock-out animal” may be generated such that gene expression is undetectable or insignificant. In another application, ectopic expression is produced by operatively inserting regulatory sequences, including inducible, tissue-specific, and constitutive promoters and enhancer elements, to direct altered spatial and/or temporal expression of an endogenous gene. Transgenic, nonhuman animals can also be produced using systems that provide regulated expression of the transgene, such as the cre/loxP (Lakso et al., PNAS (1992) 89:6232-6236; U.S. Pat. No. 4,959,317) and FLP/FRT (O'Gorman et al. (1991) Science 251:1351-1355; U.S. Pat. No. 5,654,182) recombinase systems.

In another embodiment, an engineered teleost may be generated by inducing mutations in flt1 using non-targeted (random) mutagenesis techniques, for instance, chemical-, X-ray, or transposon mutagenesis (e.g., Chen et al., Development 123:293-302 (1996); Coghill E et al., Nature Genetics 2002 30:255-256; and Kawakami K et al., Proc Natl Acad Sci USA 2000, 97:11403-11408). For example, as described in Example 1 below, teleosts are mutated using a selected mutagen (e.g. ethylnitrosourea), mutated males (P-generation) are crossed with wildtype females to generate heterozygous mutants (F1-generation) that are inbred to generate multiple heterozygous F2-carriers. These heterozygous mutants are crossed to generate homozygous mutants (F3) (Hafter P, et al. (1996) Development 123: 1-36). Homozygous mutants are analyzed for the flt1 phenotype, and genetic mapping is carried out to determine if the flt1 phenotype is caused by a mutation in endogenous flt1.

Another means for generating an engineered teleost is through nucleic acid inhibition. The nucleic acid inhibitor can be DNA, RNA, a chimeric mixture of DNA and RNA, derivatives or modified versions thereof, single-stranded or double-stranded. In one embodiment, the inhibitor is a flt1-specific antisense oligomer, preferably of length ranging from at least 6 to about 200 nucleotides. The oligomer can be modified at the base moiety, sugar moiety, or phosphate backbone. In a preferred embodiment, the antisense oligomer is sufficiently complementary to flt1 to bind to Flt1 mRNA and prevent translation.

In a preferred embodiment, the antisense oligomer is a phosphorothioate morpholino oligonucleotide (PMO). PMOs are assembled from four different morpholino subunits, each of which contains one of four genetic bases (A, C, G, or T) linked to a six-membered morpholine ring. Non-ionic phosphodiamidate intersubunit linkages join polymers of these subunits. Methods of producing and using PMOs and other antisense oligomers are well known in the art (e.g., Summerton J and Weller D, Antisense Nucleic Acid Drug Dev 1997, 7:187-95; Probst J C, Methods 2000, 22:271-281; and U.S. Pat. Nos. 5,235,033 and 5,378,841). Methods for gene inactivation in zebrafish using PMOs are well known in the art (Nasevicius A and Ekker S C, Nat. Genet. 26, 216-220 (2000). PMOs of this invention, which knock down flt1 activity, are referred to herein as “flt1 PMOs”, and are approximately 10-50 nucleotides, preferably approximately 15-40 nucleotides, preferably 20-30 nucleotides, and most preferably 21-25 nucleotides. Preferred PMOs are directed to the 5′ end of the flt1 gene such that they cover or lie upstream of the start codon. For example, preferred PMOs comprise a sequence complementary to contiguous nucleotides, preferably 10-50 contiguous nucleotides, within nucleotides 1-415 of SEQ ID NO: 1, wherein the PMO is directed to the 5′ UTR, or if it is directed to coding region (nucleotides 366 et seq.), it includes sequence complementary to the start codon located at positions 363-365. An exemplary PMO sequence is presented in SEQ ID NO: 3. As further detailed in the Examples, knock-down of flt1 using this PMO reproduced the flt1 phenotype. PMOs can also be directed to splice sites of the flt1 gene. All but three of the exon-intron boundaries of flt1 are provided in SEQ ID NOs: 3-57. For each sequence, the splice site is between nucleotides 25 and 26. In a preferred embodiment, the PMO comprises a sequence complementary to 10-50 contiguous nucleotides of any of SEQ ID NOs 3-57, and preferably includes sequence complementary to the splice site region (i.e. nucleotides 25 and 26).

Alternative nucleic acid inhibitors that may be used to knock-down flt1 activity are double stranded RNA duplexes, or “small interfering RNAs” (Elbashir S M et al., Nature 2000, 411:494-498).

Use of Flt1 Mutant Teleosts in Screening Assays

The engineered mutant teleost embryos of the invention can be used in forward and reverse genetic screens to identifying interacting genes in the flt1 pathway. As used herein, an “interacting gene” includes genes that directly or indirectly genetically interact with flt1, or genes whose protein products show direct or indirect biochemical interactions with the Flt1 protein. The engineered mutant teleost embryos can also be used to screen for pharmaceutical agents that are capable of altering the flt1 phenotype. In one exemplary genetic screen, a teleost that is a heterozygous carrier of an induced mutation of flt1 is crossed with a second teleost that is a heterozygous carrier of an induced mutation in a gene of interest. As used herein, the term “gene of interest” may be a pre-identified gene, for example, one mutated by targeted mutagenesis. Alternatively, a gene of interest may be an unidentified mutated gene, for example, generated through random mutagenesis. Further, the gene of interest is one that does not result in the flt1 phenotype when mutated in one copy (i.e. in heterozygous carriers of the mutated gene) in a wild-type flt1 background. The cross generates doubly heterozygous progeny that are heterozygous carriers of the induced mutation in flt1 and of the induced mutation in the gene of interest. The progeny are examined, preferably at 2-5 dpf, and most preferably at day 4 dpf, for the flt1 phenotype. A gene of interest is said to “interact with flt1” when a double heterozygote displays a vascular defect phenotype, including but not limited to the flt1 phenotype, and when the defect is due to the presence of both mutations. In this case, both the mutation in flt1, and in the gene of interest can be said to cause the vascular defect phenotype. If the identity of the gene of interest that causes a vascular defect phenotype is not already known, it is identified through standard mapping and cloning procedures. Vascular defects can be observed in teleost embryos using a variety of methods known in the art, such as by observation of live embryos, staining of endothelial cells, injection of dye into the circulatory system, in situ hybridization, antibody staining etc. Various methods of observing vascular defects are detailed in the examples below.

In an alternative screen to identify flt1 interacting genes, teleosts that are heterozygous carriers of an induced mutation of flt1 are self-crossed. The progeny eggs are injected with a nucleic acid inhibitor (e.g. PMO) that inhibits the function of a gene of interest, and are allowed to develop into embryos under standard conditions. In this type of screen, the gene of interest is one that does not result in the flt1 phenotype when knocked-out in an otherwise wild-type background, i.e. it does not independently cause an flt1 phenotype. The vasculatures of the injected embryos are examined for defects. If the gene of interest is not an interacting gene, approximately 25% of the embryos (i.e. those homozygous for the flt1 mutation) will display the flt1 phenotype, according to normal Mendelian segregation. If the gene of interest is an interacting gene, then significantly greater or fewer than 25% of the embryos will display the flt1 phenotype. By “significantly greater or fewer”, it is meant that there is a statistically significant difference in the percentage of progeny that exhibit the flt1 phenotype in comparison to the percentage of progeny that exhibit the flt1 phenotype when heterozygous carriers of a mutation of flt1 are self-crossed. For example, if the gene of interest promotes sprouting of vessels via the flt1 pathway, inhibition of the gene in heterozygous flt1 mutant embryos may induce a similar vascular defect as observed in homozygous flt1 mutant embryos, resulting in greater than 25% of the injected embryos displaying vascular defects similar to the homozygous flt1 mutant embryos. In one example, the flt1 phenotype will be observed in approximately 75% of injected embryos, representing the approximately 25% that are homozygous for the flt1 mutation, plus the approximately 50% that are heterozygous for the flt1 mutation, in which loss of function of the interacting gene causes a flt1 phenotype. If the gene of interest is an antagonist of flt1 mediated vessel sprouting, then loss of function of the gene of interest may result in a rescue of the mutant phenotype. Therefore, either less than 25% of the injected embryos will display the flt1 phenotype or the flt1 phenotype will be partially reverted into the wildtype phenotype.

In another type of screen to identify flt1 interacting genes, nucleic acid inhibitors are used to knock down activity of flt1 and/or a gene of interest. In one example, a flt1 PMO is injected into wild-type teleost eggs together with a PMO directed to a gene of interest. The injected eggs are allowed to develop into embryos, preferably to 2-5 dpf, and are most preferably examined at 4 dpf. As controls, wild-type teleost eggs are injected with flt1 PMO alone or the PMO directed to the gene of interest alone (i.e. “single injection controls”). If the gene of interest is an interacting gene, the resulting phenotype will be different from the phenotypes of the single injection controls. If the gene of interest is an antagonist of flt1 function, the vascular defect will be at least partially rescued, relative to injections with flt1 PMO alone. If the gene of interest is an agonist of flt1 function, then a more severe vascular defect phenotype will result compared with injections with flt1 PMO alone. In another example of a screen that uses nucleic acid inhibition, a flt1 PMO is injected into teleost eggs derived from a mating of a heterozygous mutant carrier of a defect in a gene of interest with a wildtype teleost. The gene of interest is identified as interacting with flt1 if a more or less severe vascular defect phenotype is observed in 50% of the embryos as compared to the flt1 phenotype.

In a screen to identify pharmaceutical agents capable of altering the flt1 phenotype, teleosts that are heterozygous carriers of an induced mutation of flt1 are crossed. The progeny eggs or early embryos are contacted with test substances, for example by injection or soaking. The vasculature of the embryos is examined for defects. If fewer than 25% of the offspring display the flt1 phenotype, the test substance is identified as a candidate pharmaceutical agent that promotes sprouting of vessels, and that is capable of reverting an flt1 mutant phenotype into a wildtype phenotype or a phenotype that has fewer vascular defects than the flt1 phenotype.

### EXAMPLES

Identification of Zebrafish Mutations with Specific Vascular Defects by Alkaline Phosphatase Staining

In a large scale genetic screen we examined the F3-progeny of zebrafish males mutagenized with ethylnitrosourea (ENU). Maintenance and mutagenesis of zebrafish were essentially done as described (Haffter P, et al., Development (1996) 123:1-36.). The strain used for mutagenesis was Tübingen. Larvae were fixed at 4 dpf in 4% para-formaldehyde in phosphate-buffered saline (PBS) for 30 minutes. Fixed larvae were dehydrated in methanol and stored over night at −20° C. After permeabilization in acetone (30 minutes at −20° C.) embryos were washed in PBS and incubated in the staining buffer (100 mM Tris-HCl [pH 9.5], 50 mM MgCl₂, 100 mM NaCl, 0.1% Tween-20) for 45 minutes. Staining reaction was started by adding 2.25 μl nitro blue tetrazolium (NBT, Sigma) and 1.75 μl 5-bromo-4-chloro-3-indolyl phosphate (BCIP, Sigma) per ml of staining buffer (stock solutions: 75 mg/ml NBT in 70% N,N-dimethylformamide, 50 mg/ml BCIP in N,N-dimethylformamide).

The fixed specimens were scanned for changes in blood vessel formation. By this means, we scored 4,521 mutagenized genomes and identified more than 700 mutants. Two of these mutants, referred to herein as Schwentine1 and Schwentine2, displayed identical changes of their circulatory system. Complementation analysis of heterozygous carriers of the two mutations revealed that these two mutations are allelic. In mutant embryos, the overall morphology including the circulation in the dorsal aorta and posterior cardinal vein appeared to be normal up to 4 dpf. After this time point a heart edema of increasing size formed and the embryos became necrotic. The swim bladder inflated only rarely and the mutant larvae died approximately at 7 dpf.

The alkaline phosphatase staining revealed specific defects of the vasculature Referring to FIG. 1b, intersegmental vessels (Se) spanned the whole lateral aspect at only a few of the somite boundaries of the larva, while in the others the intersegmental vessels just reached the horizontal myoseptum or were missing altogether. The parachordal vessels (PAV) were well developed in mutants and connected the shortened the intersegmental vessels. In the mutants, the subintestinal vein (SIV) was thin and often interrupted. The number of branches that spanned the yolk was reduced. The changes of the Se and the SIV were variable. For example, both the number and the identity of the missing or shortened Se differed from one mutant larva to another. Starting at 3 dpf in approximately 30% of the mutant larvae, accumulations of erythrocytes could be observed either around the eyes or around the developing swim bladder.

Analysis of Vascular Defects by Confocal Microangiography

We used confocal microangiography techniques as described (Weinstein et al, supra) to analyze the vascular defects of the Schwentine1 mutant in more detail and examined the functionality of the blood vessels. Optical sections were taken using a Leica TCS NT confocal microscope. At 24 hpf the circulation in zebrafish is established and at this stage of development Schwentine mutant and sibling larvae were indistinguishable. The main components of the circulatory system in the trunk and tail, i.e. the heart, the aortic arches, the dorsal aorta, the caudal artery, the caudal vein, the posterior cardinal vein, and the common cardinal veins formed and functioned normally. The first alterations in the mutants became visible at 2 dpf. At this stage of development in the wild type, intersegmental vessels have luminized at every somite boundary. In Schwentine mutant embryos, however, only about 50% of somite boundaries had at least one out of two bilateral intersegmental vessels that reached the most dorsal region. At the other somite boundaries, the intersegmental vessels were absent or stopped half way through the lateral aspect of the trunk at the level of the horizontal myoseptum. At a single somite boundary, the length of the left and right intersegmental vessels often varied. Wherever full-length intersegmental vessels were present in the mutant embryos, they were connected by a dorsal longitudinal anastomotic vessel as in the wild type. All these vascular defects remained and could clearly be seen at 4 dpf (see FIGS. 1a, b). In contrast to the alkaline phosphatase staining, only the most proximal parts of the subintestinal vein were detectable by the injection of a fluorescent dye proving the main part of this vessel to be without continuous circulation. Also, the pectoral fins lacked the vascular loop consisting of the pectoral artery and the pectoral vein.

The head vessels in Schwentine mutant embryos developed normally up to 1.5 dpf establishing all main routes of blood supply. After this time point in the wild type, the central arteries start to sprout and penetrate into the brain substance forming a complex network. In Schwentine mutant embryos none of the central arteries could be detected in microangiographies. At 4 dpf, still no central arteries had formed while the vessels with a large diameter were normally developed (see FIGS. 5a, 5b). A remarkable exception was the lateral dorsal aorta. In the wild type, the section between the efferent artery of the first branchial arch and the caudal division of the internal carotid artery consists of a single vessel without any branches. In contrast, in Schwentine mutant larvae a vascular plexus of variable size formed (see FIGS. 6a, 6b).

The phenotypic analysis of Schwentine1 has shown that the formation of the initial circulatory loops in trunk and tail occurs normally. At the onset of circulation around 24 hpf mutant and sibling larvae are indistinguishable. Whole mount in situ hybridizations using markers for angioblasts (flt1, scl) confirmed that the assembly of angioblasts is not affected in mutant embryos. It is only after the formation of the dorsal aorta and posterior cardinal vein that the first alterations become visible on a molecular level. The sprouts of the Se cannot be detected in flt1 in situ hybridizations of flt1 mutant embryos. This defect is mirrored on a morphological level, since in flt1 mutant embryos the Se fail to form in approximately half of the somite boundaries. The other vessels that are either affected in their formation or function are the central arteries of the brain, the blood supply of the digestive system (most prominent the SIV), and the pectoral artery and vein. As discussed earlier these vessels have been implicated to involve sprouting during their generation. For the acquisition of blood vessels in quail limbs it has been shown that angiogenesis is the driving force (Jotereau F V and Le Douarin N M, Dev Biol (1978) 63:253-265; and Pardanaud L, et al., Development (1989) 105:473-485). A failure in sprouting might also explain the observed accumulations of erythrocytes around the swim bladder, since the formation of the blood vessel plexus on the swim bladder takes place around 3 dpf. The blood pools around the eye might involve the formation of lacunae in the choroid plexus comparable with the changes in the lateral dorsal aorta.

Molecular Cloning of Zebrafish Flt1 Gene

A mapping strain was generated by crossing a Schwentine TUE male to a WIK female. The progeny was grown to maturity and diploid mutant larvae were used for linkage analysis. The Schwentine allele was mapped to linkage group 14 in an interval between the microsatellite markers z1226 and z36206. The remaining markers in this region were not informative. A comparison with the radiation hybrid map showed that a fragment published as kdr (flk1, vegfr2) ((Fouquet B, et al., supra; Liao et al., supra; Sumoy L, et al., supra; and Thompson M A, et al., supra) mapped in this interval. We assembled a contiguous stretch of genomic DNA covering the whole locus and generated a simple sequence length polymorphism (SSLP) marker in an intron of the gene. This marker called HH01 showed no recombinant in 3122 meioses. In parallel to our mapping efforts we performed 5′RACE for the gene published as kdr to obtain the full-length sequence. The 5′ end of flt1 was amplified using a primer in the published partial sequence (Genbank accession number: AF180354) using the Marathon™ cDNA amplification kit, and Advantage™2 polymerase mix (both Clontech Laboratories, Inc.). Using the BLAST and ClustalW programs, a comparison of the amino acid sequence with VEGF receptor sequences of other species clearly showed that the gene previously published as kdr encodes the zebrafish orthologue of the mammalian flt1 gene (FIG. 3b). Therefore we suggest changing the name of this gene to flt1.

Nucleic Acid Inhibition of Flt1

An antisense morpholino oligonucleotide (PMO) of the sequence 5′-CCGAATGATACTCCGTATGTCAC-3′ (SEQ ID NO:3), which targets the 5′UTR of the flt1 messenger RNA, was dissolved at a concentration of 3 mg/mL in injection buffer (0.4 mM MgSO₄, 0.6 mM CaCl₂, 0,7 mM KCl, 58 mM NaCl, 25 mM Hepes [pH 7,6]). A total of 1.5 nL (=4.5 ng) was injected into zebrafish embryos at the 1-cell stage. For controls, the inverse sequence, or a version with four mismatching basepairs, was injected. The mutant vascular phenotype was never observed in controls. Larvae injected with the PMO directed to flt1 displayed no obvious alterations of their overall morphology. Alkaline phosphatase staining, however, revealed exactly the same vascular defects as observed in Schwentine mutant larvae. This finding together with the linkage analysis strongly suggested to sequence the flt1 cDNA from mutant larvae of both alleles. Living larvae of heterozygous carriers of Schwentine1 & 2 mutants were sorted according to their vascular phenotype at 4 dpf. RNA was isolated and transcribed into cDNA using SuperScript™II reverse transcriptase (Life Technologies). Three overlapping fragments covering the whole flt1 gene were amplified using ELONGASE® enzyme mix (Life Technologies) and sequenced. The confirmation on genomic DNA was done by amplifying a 200 bp fragment and subsequent sequencing. We found point mutations in the open reading frame of both alleles, that resulted in stop codons causing truncation of the Flt1 protein, to contain only four and five extracellular Ig-like domains instead of the seven Ig-like domains of full-length Flt1. Further, the transmembrane domain and the intracellular part containing the tyrosine kinase domain were lacking in both alleles. These single base pair changes were confirmed on genomic DNA of 20 phenotypically mutant and 20 sibling larvae for each allele.

The Expression of Flt1, but not of Other Markers for Vasculogenesis and Hematopoiesis is Affected in Flt1 Mutant Embryos

To analyze the consequences that a mutant Flt1 receptor might have on the expression of genes that are involved in hematopoiesis and vasculogenesis we performed whole mount in situ hybridizations. The messenger RNAs of scl, gata1, fli1, vegf and flt1 were detected in the progeny of heterozygous t²⁰²⁵⁷ carriers. After staining, the embryos were examined for alterations of the expression pattern and subsequently all embryos were genotyped one by one. At the 7 somite stage all embryos with a mutant genotype (n=10) displayed a strong reduction of flt1 expression. In contrast, the expression of scl, fli1, vegf and gata1 were indistinguishable between sibling and mutant larvae at this stage of development (n=80 for each probe). At 28 hpf in all mutant embryos (n=16) the flt1 expression was still down regulated while in sibling larvae (n=56) the flt1 staining outlined the circulatory system. The expression of scl, gata1, and vegf was unaltered. The fli1 staining in mutant embryos demarcated the main circulatory routes as it did in the siblings. The only exception was the sprouts of the forming Se that were absent in 60% of the mutant embryos (n=17). The decrease of flt1 mRNA has no effect on the expression level or pattern of other genes involved in vasculogenesis or hematopoiesis up to 24 hpf. So despite the expression being limited to angioblasts and endothelial cells, and the early onset of transcription, flt1 is not necessary for the control of blood cell formation or the establishment of the initial circulatory system.

Flt1 Function is Necessary for the Formation of Additional Vessels upon Vegf₁₆₅ Overexpression

The overexpression of the Flt1 ligand vegf₁₆₅ stimulates the formation of surplus blood cells. To test whether the Flt1 receptor is necessary for VEGF signal transduction we overexpressed vegf₁₆₅ ectopically. For this purpose we injected a DNA construct containing the vegf₁₆₅ cDNA under the control of the CMV promoter into embryos derived from heterozygous Schwentine1 carriers. The coding region of vegf₁₆₅ was cloned into the EcoR1 sites of plasmid pCS2+ (Liang D, et al., Biochim Biophys Acta (1998) 1397:14-20). The construct was cut with SalI/NotI to obtain a fragment containing the CMV promoter+vegf₁₆₅+polyA signal. The gel purified DNA was diluted with water to a concentration of 20 ng/μL. Approximately 3 nL of this solution was injected into the progeny of heterozygous Schwentin1 carriers at the one cell stage. After documentation the specimen were digested to isolate genomic DNA and the marker HH01 was used to determine the genotype. In the controls (uninjected or injected with a fragment containing only the CMV promoter+polyA signal) the formation of additional sprouts was never observed. The DNA injection led to a high expression level of vegf₁₆₅ in the yolk while the expression was unaltered in the rest of the embryo.

In a first set of experiments we investigated the impact of VEGF overexpression on hematopiesis. The eggs of four different clutches were injected with 75-100 pg of the DNA construct (n=145). In 26.6±16.2% (n=41) of the embryos the injection led to an increase of blood cells pooling in the tail. Subsequent genotyping showed that a quarter of the affected embryos had a mutant genotype. So overexpression of vegf₁₆₅ influenced hematopoiesis both in sibling and flt1 mutant embryos. Thus, Flt1 function is not required for hematopoietic VEGF signaling.

We further asked what effects the ectopic expression of vegf₁₆₅ might have on the formation of blood vessels. A total of 432 embryos from 10 different clutches were injected with 50 pg of the DNA construct, fixed at 4 dpf, and stained to visualize the vasculature. Almost all vessels were unchanged. Only the SIV showed a clear alteration resulting in the formation of additional vessels between the SIV and the common cardinal vein. This effect could be observed in 16.5±10.5% of the larvae, while 24.0±6.0% showed a mutant SIV. Genotyping revealed that all specimen with additional vessels had a sibling genotype (n=52). In contrast mutant larvae never formed such additional vessels, but the SIV remained thin and the number of branches was still reduced (n=79). Thus overexpression of vegf₁₆₅ in the yolk sac is sufficient to cause the outgrowth of additional vessels in wild-type embryos. Such a response to a VEGF stimulus has been reported earlier from quail, chicken and frog (Flamme I, et al., Dev Biol (1995) 171:399-414; Wilting J. et al., Dev Biol (1996) 176:76-85; and Cleaver O, et al., Dev Dyn (1997) 210: 66-77). However, in the absence of a wild-type Flt1 receptor, i.e. in the flt1 mutant larvae, additional blood vessels have never been observed. These results confirm, that zebrafish flt1 is specifically necessary for the sprouting of new blood vessels, while the VEGF signal for increased hematopoiesis is relayed independently. The loss of VEGF function has been studied in gene knock down experiments (Nasevicius A, et al., supra). Embryos injected with antisense morpholino oligonucleotides lack the whole vasculature including the dorsal aorta and the posterior cardinal vein. This underlines the essential role of VEGF in zebrafish vasculogenesis. On the other hand it indicates that there must be at least a second VEGF receptor in zebrafish having a pivotal role in VEGF dependent vasculogenesis, since the loss of flt1 function does not affect vasculogenesis. Our results show that flt1 has quite a different role in zebrafish development than it has in mice. The vascular defects in zebrafish flt1 mutants do not resemble any of the described phenotypes of VEGF receptor knock outs.

What is claimed is:

1. An engineered mutant teleost embryo having reduced flt1 activity that causes a phenotype of normal assembly of main circulatory routes and a reduction in sprouted blood vessels.

2. The teleost embryo of claim 1 that is a homozygous carrier of an induced mutation of flt1.

3. The teleost embryo of claim 1 that contains an exogenously added nucleic acid inhibitor that specifically inhibits flt1.

4. The teleost embryo of claim 3 wherein the exogenously added nucleic acid inhibitor is an antisense phosphoramidate morpholino (PMO).

5. The teleost embryo of claim 1 that is a zebrafish.

6. A method for identifying a gene that interacts with flt1 comprising:
a) crossing a teleost that is a heterozygous carrier of an induced mutation of flt1 with a second teleost that is a heterozygous carrier of an induced mutation in a gene of interest to generate doubly heterozygous progeny that are heterozygous carriers of the induced mutation in flt1 and of the induced mutation in the gene of interest, and
b) examining vasculature of the doubly heterozygous progeny, wherein a double heterozygote that displays a vascular defect phenotype identifies the gene of interest as a gene that interacts with flt1.

7. The method of claim 6 wherein the induced mutation in the gene of interest is targeted.

8. The method of claim 6 wherein the induced mutation in the gene of interest is random.

9. A method of identifying a gene that interacts with flt1 comprising:
a) crossing teleosts that are heterozygous carriers of an induced mutation of flt1, to generate fertilized eggs;
b) injecting the fertilized eggs with a nucleic acid inhibitor that specifically inhibits a gene of interest;
c) culturing the eggs under conditions that allow formation of teleost embryos, and
d) examining vasculature of the teleost embryos;
wherein the gene of interest is identified as a gene that interacts with flt1 if the percentage of teleost embryos that exhibit an flt1 phenotype is significantly higher or lower than 25%.

10. The method of claim 9 wherein the molecule is a PMO.

11. A method for identifying a gene that interacts with flt1 comprising:
a) injecting a flt1 PMO together with a PMO directed to a gene of interest into wild-type teleost eggs,
b) culturing the eggs under conditions that allow formation of teleost embryos, and
c) examining vasculature of the teleost embryos,
wherein the gene of interest is identified as a gene that interacts with flt1 if the teleost embryos exhibit a phenotype that differs from phenotypes exhibited by single-injection control teleost embryos.

12. A method for identifying a gene that interacts with flt1 comprising:
a) injecting a flt1 PMO into teleost eggs derived from a mating of a heterozygous mutant carrier of a defect in a gene of interest with a wildtype teleost,
b) culturing the eggs under conditions that allow formation of teleost embryos, and
c) examining vasculature of the teleost embyos,
wherein the gene of interest is identified as a gene that interacts with flt1 if a more or less severe vascular defect phenotype is observed in 50% of the embryos as compared to a flt1 phenotype.

13. A PMO that specifically inactivates a teleost Flt1 gene, said PMO comprising a nucleotide sequence of 10-50 nucleotides that are complementary to contiguous nucleotides within a nucleotide sequence selected from the group consisting of any one of SEQ ID NOs:3-57, and nucleotides 1-400 of SEQ ID NO: 1.

14. A PMO of claim 13 having a nucleotide sequence complementary to 21 to 25 contiguous nucleotides within nucleotides 1-365 of SEQ ID NO: 1.

15. A PMO of claim 13 that comprises SEQ ID NO:3.


SEQUENCE LISTING (57 sequences)

SEQ ID NO: 1 - DNA, 5789 bp, Danio rerio
tggtgtgaagatagataaatggctgttacaccttcactacatagtgtagttttggaaatttcattcattcggttttaggaaggacggacagcggtcgcacctgccctcgggagttgtggagtcagtggagtttctccattcgtcttagtaccggggcaggcgcagtcctgcgcgatactttacagtcactgagaggtgtttaactattaacacgtcaccgaagaaccatctctatcatatcaacaggaactataagcatattatcatacatcaagagaaattaagttctggactactgcagccgcatccgaacgtgaagtgacatacggagtatcattcgggacgttattttaacagacaaaatgactcctcttaaaacctcagtcaaagccttcttcactcttcacgtgctttttagttgtatttcacatggtttggtagagggatctcgtctgcctgatccacaactgcttcctgatggagatacacaccttcagcatgttggtgggacactcacactcatttgcagaggatcaactgcactgcactggaggctggctagcagaaatgtgtcatcggtacgcattgagtcgtgtgaggagagactgcacaagcactgcagcaagctagtcatacacaacttgaggcacaacgacactggcatctactcatgcagtcacaagaagtccagcgatcatgaagtgtccacatatgtttttgttaaagatccccatcatccatttgtggaggcttacagtttaccacatccgctttttgcatatcgaaatgatccatattttgtggtcccctgtagaacaacatacccaaaccaaaacgttatccttgagacgcagatgaatcctatggcagatgatgttaaaagaggggtacagtgggatccaaaaaaaggtttcacggtccctttaaaaccttatgacagctaccatctaattacatgcttgaccagggtggataacgcagaattttcatcagtctacctacttaaaaggctaacaatggaaattaaaaaccttgccatcgaaccagaaagaccaagagtacttgtcggtgacacccttcatccttcaactgcttctgcagagaccacttacaatggctggattcactttgagtgggagtttcataaggagcggatcaatcgtactcaccattttagcaccacaccggtccaacttgcccagattatggtgatgtctaaagcattaatcgtgcctaatgtgacaatggaggacaagggcacatacacgtgcacaggatcaattgaatttaaaaagcttcaaatgtcaaccaaagttattgtttatgaacatccattcctcaatgttactcataataaacgtaaattcacctctactgtagaaggtagaagggtgcagtttgagcctcgggtcaatgctgttcctgcgcccgacagagtcttgtggtataaagatggagtggccatttctgaaaattccacatgttacgaaactgcaggctacaacctgaccataaaacaagtgaggcagaaggatgctgggattttcactatcgctctgtcaaaccaggagaggggtctctacagaaacatcagttacaagcttgaagtcagagtgaagccaaagatctttgaagaggatgtggctccagcaggtccccagaccttcagatatgaccagaggcacaagctcacctgcactgcatttggtattcccatgccgaacattacctggttttggcagccgtgtgaccccagtgctaatcttacagaatgcaaactttacactgatcctcttcccattgaaaacgttgatgaccattttcctcaaaatccgataaaggatgtaaacagcaaggttggattgttaaaaagtaaaaaccggactataagtaccctggtggtgaaaacagctaatgtgtctggggtatactcctgtacagcaaggaatgaacttggcaaccggaccatgagaatccctttttatgtggatgatcacccacagccttttgagattgagccctccactgcggttgctggagatgacattacattgacttgcagaggcacaagatacctctacgataggctgacttggtatgatcctctgggccataaggtgcccaaagatgaaaccactctgcggattgagccctacactatatctttgtcaatcaagctgcccaatgtttccagaaaccatacactcggctatgagtgccaggccttaaacatcaacaccaataaagtggtcaatgtaacatctgctttgactattgatgagaggcaagggccctggttaatgcagaatctgaccaatcaggatgtaaacagcagcagcaccctaacactggcctgcttggcgtatggagttccagcaccctttatcacatggtacaaagacaagactcctgtcacagagggaccaggaatcactttaaaagatgatggcactctaattattgaaagggtgaagaaggacgatgagggcatttatgaatgtcgtgctagcaatgatggaggggaggcaaaaaccagtgcggtcattactgtagtaggagaagatggtaaaccaaatattgaggtcatcatcttggtgtcgactggagcagcagcaacattcctgtggattatgctcattctctttatccgcaaattaagaaagccaagttcagcagatttaaagacagggtacctgtccatcatcatggatcctgaacagatgcctctcgatgagcagtgtgaccgtctaccatatgacagcaacaaatgggagtttcctcaagatcgcctcagactcggtaaaactttgggccatggagcatttggaaaagttgtagaggcctctgcatttggcattgacaagatttcaacatgcaaaacagtggctgtgaaaatgctgaaagtgggagcaacaaataacgaatggagagccttaatgtctgaactgaagatactgatccatattgggcatcatctcaatgtggttaacctgctaggagcctgcacaaagcgtggcggccccctaatgattatcgtggaattctgcaagtatggaaatctttccaactacctgagaagtaagagaggtgactttgtggtttacaagtctcaggacggtaaggctgtgcgttccagctcaggctgtgatctgagcgagctcatcaagcgcaggttggagagcgtggcgagcaccggaagttcagccagctccggcttcattgaagataagagctactgcgactcggaggaagaggaggaagagcaagaggatttgtataagaaagtgctcacattggaagacttgatttgctacagctttcaagtggctaaaggcatggagttcttggcttcgagaaaatgtatccaccgtgatctggctgcacgtaacatcctgctgtctgaaaacaatgttgtgaagatttgcgattttggacttgcaagagatgtatacaaggacccagactatgtccgcaaaggagacgctagacttcccttaaagtggatggcgccagaggccatttttgacaagatctatactactcaaagtgacgtgtggtcttttggagtgcttatgtgggagatcttctctcttggtgcctccccttaccctggcttacacattgatgaggaattctgctgccgactgaaggagggcactaggatgaaagctcctgagtactcctcctctgaaatatatcagaccatgttggactgctggcatggagaaccatctcagaggcccactttcacagagctggtggagaggctaggagatttgctacaggctagtgtgcagcaggagggaaagcactacatcccgatcaacacggccctcttgaccaaagcagacccctcaaaccagagtcccacagaggagacctccacacgaccagtctctctcagagactctgggacggcttggaacatcaagatccgcccagagagtgtgaagaccttcgatgaagtaatcctggagaacggaaccaacaagatccacgagggtgggcagtcagacagtgggataggactatcttcagatgacctgaagacgctgaagcggctggagtccctggcccgaccccggagtttcatgtcccgagcgatgaagaggaaaagtaaggagtcagttctgctggagggcgaaatggacaaatacccaccgcttgttccctcactgagtctggaggactcgtcccttgactcggagatggagtgtcacagtcctcctccagactataactatgtggttcgttactccacaccacccgtctgacatgctctgcgctggaaaagccgccagaagtctgatcggtccattctgtctcattctgtctctgtctctacttttatttttcccacacatggtcattcagagattcacatttcctgtacacttctgtcaagttagtgaaggaatacagtcggctgccactcatatctgtggtagtgtaagattcaaatggaccagctcaggtatttctgctgtctactgtgaacacaacaggcacattttattacactaacagcactgtttccccattgaaaatgcattcttcagtcagaatggtggctaattatgaaatcattagttcggtggttagagcgagaggacaaaggcaatccttattttatttctagataaatattttattgaaatgaaactggaattttggttatgtgatattttattttgctaaacgtattctttcagagctcgctaaagcttcttctttgaaagggaaaaactatttttctttagaacagatttcactcaaactttcagacaagctgtcaaacaaagaccacagatcatgaataatttaacatgcggtcagaagctccttcccagatctccatcacggcacattgatcatcagcatacttatgatgcacattgtgtttccaggttcagtcgcaccagtctgagggctgatttcctgagtcttactcggtaaagctaaaatgaatcacttctgttcccgcttgtgctatttttatctggtaaacatcttataatatggttaatttagttaaaattcagtttaagatcttcaacttgcttatggatgccacaacattcagtccatcactgtgaaagtaaattactcatgccaatttcataggaaacattcagttaattattgatcaaatgtggatgatataaaggccttgctctgttcaatacacaacagttacactgcatcgtgtactttccatatttatttcgtttacactactgtatacataacagcacgtaatatgttgtgcagctattaaccagaggagcgtatcctgttactgattcagtagtcgtttgtgctaaaatgctactaaagcttgcagggcaggctatgatattgtcaacaggctggtcactatattaatgttcatgaaaatgatatgcacacatgatgtaggtgctatcagtattaactccttaacatctgtatgtacgtaagggaaaggttgggagattatcgtattggttaagccagattatattgactgtgtaaagggccctttgctaaccgttatcgtgtttaaatcccagtattacattcatagctgatgatgtactgttttccaaataatgaaatgtgtatatactgccaaatgatgctttccattatgttgttattttgactgatatttgtgaagcaaaattgtatgcactgatattttctacacgtatgtattttttgtattataactgtattttaatattaaccaagcataaataaagatctatatatttatatttaaaaaaaaaaaaaaaaaaaaaaaaaaa

SEQ ID NO: 2 - protein, 1302 aa, Danio rerio
MTPLKTSVKAFFTLHVLFSCISHGLVEGSRLPDPQLLPDGDTHLQHVGGTLTLICRGSTALHWRLASRNVSSVRIESCEERLHKHCSKLVIHNLRHNDTGIYSCSHKKSSDHEVSTYVFVKDPHHPFVEAYSLPHPLFAYRNDPYFVVPCRTTYPNQNVILETQMNPMADDVKRGVQWDPKKGFTVPLKPYDSYHLITCLTRVDNAEFSSVYLLKRLTMEIKNLAIEPERPRVLVGDTLHPSTASAETTYNGWIHFEWEFHKERINRTHHFSTTPVQLAQIMVMSKALIVPNVTMEDKGTYTCTGSIEFKKLQMSTKVIVYEHPFLNVTHNKRKFTSTVEGRRVQFEPRVNAVPAPDRVLWYKDGVAISENSTCYETAGYNLTIKQVRQKDAGIFTIALSNQERGLYRNISYKLEVRVKPKIFEEDVAPAGPQTFRYDQRHKLTCTAFGIPMPNITWFWQPCDPSANLTECKLYTDPLPIENVDDHFPQNPIKDVNSKVGLLKSKNRTISTLVVKTANVSGVYSCTARNELGNRTMRIPFYVDDHPQPFEIEPSTAVAGDDITLTCRGTRYLYDRLTWYDPLGHKVPKDETTLRIEPYTISLSIKLPNVSRNHTLGYECQALNINTNKVVNVTSALTIDERQGPWLMQNLTNQDVNSSSTLTLACLAYGVPAPFITWYKDKTPVTEGPGITLKDDGTLIIRVKKDDEGIYECRASNDGGEAKTSAVITVVGEDGKPNIEVIILVSTGAAATFLWIMLILFIRKLRKPSSADLKTGYLSIIMDPEQMPLDEQCDRLPYDSNKWEFPQDRLRLGKTLGHGAFGKVVEASAFGIDKISTCKTVAVKMLKVGATNNEWRALMSELKILIHIGHHLNVVNLLGACTKRGGPLMIIVEFCKYGNLSNYLRSKRGDFVVYKSQDGKAVRSSSGCDLSELIKRRLESVASTGSSASSGFIEDKSYCDSEEEEEEQEDLYKKVLTLEDLICYSFQVAKGMEFLASRKCIHRDLAARNILLSENNVVKICDFGLARDVYKDPDYVRKGDARLPLKWMAPEAIFDKIYTTQSDVWSFGVLMWEIFSLGASPYPGLHIDEEFCCRLKEGTRMKAPEYSSSEIYQTMLDCWHGEPSQRPTFTELVERLGDLLQASVQQEGKHYIPINTALLTKADPSNQSPTEETSTRPVSLRDSGTAWNIKIRPESVKTFDEVILENGTNKIHEGGQSDSGIGLSSDDLKTLKRLESLARPRSFMSRAMKRKSKESVLLEGEMDKYPPLVPSLSLEDSSLDSEMECHSPPPDYNYVVRYSTPPV

SEQ ID NO: 3 - DNA, 50 bp, Synthetic
gtgctttttagttgtatttcacatggtaagtgtgcgctttttgtgattat

SEQ ID NO: 4 - DNA, 50 bp, Synthetic
gtgttctattatttgttttctgtaggtttggtagagggatctcgtctgcc

SEQ ID NO: 5 - DNA, 50 bp, Synthetic
gtgggacactcacactcatttgcaggtactgcatccatctactgtcccac

SEQ ID NO: 6 - DNA, 50 bp, Synthetic
ttgtttttgttgtgacttccaatagaggatcaactgcactgcactggagg

SEQ ID NO: 7 - DNA, 50 bp, Synthetic
gtcttgtgttatatttaaatcacagatccccatcatccatttgtggaggc

SEQ ID NO: 8 - DNA, 50 bp, Synthetic
aaaccaaaacgttatccttgagacggtaagtattttatgttagcaacact

SEQ ID NO: 9 - DNA, 50 bp, Synthetic
atataaaatctctcttgttttgcagcagatgaatcctatggcagatgatg

SEQ ID NO: 10 - DNA, 50 bp, Synthetic
tcagtctacctacttaaaaggctaagtaagtcattttaattgctaaaatg

SEQ ID NO: 11 - DNA, 50 bp, Synthetic
aattaatgtacttttctctttgtagcaatggaaattaaaaaccttgccat

SEQ ID NO: 12 - DNA, 50 bp, Synthetic
tgagtgggagtttcataaggagcgggtgagtaactgagagcctttctaaa

SEQ ID NO: 13 - DNA, 50 bp, Synthetic
tgatattaatcctctgtgtttgcagatcaatcgtcctcaccattttagca

SEQ ID NO: 14 - DNA, 50 bp, Synthetic
atgtcaaccaaagttattgtttatggtgaggatgttgtgacactgcaata

SEQ ID NO: 15 - DNA, 50 bp, Synthetic
atttaatagatttttctctctacagaacatccattcctcaatgttactca

SEQ ID NO: 16 - DNA, 50 bp, Synthetic
ttcctgcgcccgacagagtcttgtggtaagtttgtgctttgcattataag

SEQ ID NO: 17 - DNA, 50 bp, Synthetic
atcattttgctgtcaatttgtataggtacaaagatggagtggccatttct

SEQ ID NO: 18 - DNA, 50 bp, Synthetic
atcagttacaagcttgaagtcagaggtaatgccgtgaaaaacaataagaa

SEQ ID NO: 19 - DNA, 50 bp, Synthetic
actaatgttctgtatatgcacacagtgaagccaaagatctttgaagagga

SEQ ID NO: 20 - DNA, 50 bp, Synthetic
gtgaccccagtgctaatcttacagagtaggtcaatcaatgttcacattta

SEQ ID NO: 21 - DNA, 50 bp, Synthetic
gcataacgacggctaatctctccaggatcctcttcccattgaaaacgttg

SEQ ID NO: 22 - DNA, 50 bp, Synthetic
tggattgttaaaaagtaaaaaccgggtatgtattatgtgcattattttgt

SEQ ID NO: 23 - DNA, 50 bp, Synthetic
taataatcttttctcatcctcttagactataagtaccctggtggtgaaaa

SEQ ID NO: 24 - DNA, 50 bp, Synthetic
atgagaatccctttttatgtggatggtatgttagtcattttatatgtact

SEQ ID NO: 25 - DNA, 50 bp, Synthetic
tcttattctcttttatctttactagatcacccacagccttttgagattga

SEQ ID NO: 26 - DNA, 50 bp, Synthetic
gtaacatctgctttgactattgatggtgagttttatatatcaagcgccaa

SEQ ID NO: 27 - DNA, 50 bp, Synthetic
tgcaactatccgttttctgactcagagaggcaagggccctggttaatgca

SEQ ID NO: 28 - DNA, 50 bp, Synthetic
aagactcctgtcacagagggaccaggtgagacaacatgtattaatgcgat

SEQ ID NO: 29 - DNA, 50 bp, Synthetic
atttttcttttaataatgttctcaggaatcactctaaaagatgatggcac

SEQ ID NO: 30 - DNA, 50 bp, Synthetic
accagtgcggtcattactgtagtaggtgagtagttgaataaagacaaccc

SEQ ID NO: 31 - DNA, 50 bp, Synthetic
gaaaacatgctgtttctgcacacaggagaagatggtaaaccaaatattga

SEQ ID NO: 32 - DNA, 50 bp, Synthetic
tctctttatccgcaaattaaggaaggtaagcactttgctgttattgtact

SEQ ID NO: 33 - DNA, 50 bp, Synthetic
gccaatttttttattattagaccagccaagttcagcagatttgaagacag

SEQ ID NO: 34 - DNA, 47 bp, Synthetic
gtttcctcaagatcgcctcagactcgtagttcaaagatccgggttcc

SEQ ID NO: 35 - DNA, 50 bp, Synthetic
tctctttctctctctttttctaacaggtaaaactttgggccatggagcat

SEQ ID NO: 36 - DNA, 50 bp, Synthetic
acagtggctgtgaaaatgctgaaaggtaactgtaacaggttatctttagt

SEQ ID NO: 37 - DNA, 50 bp, Synthetic
ctccaacttcctctacgcttaacagtgggagcaacaaataacgaatggag

SEQ ID NO: 38 - DNA, 50 bp, Synthetic
ctaggagcctgcacaaagcgtggcggtaagaggaggatgataagattaga

SEQ ID NO: 39 - DNA, 50 bp, Synthetic
aacaattggtttgatttacacttaggccccctaatgattatcgtggaatt

SEQ ID NO: 40 - DNA, 50 bp, Synthetic
gagaggtgactttgtggtttacaaggtacgcagtgacgcctcctgttcat

SEQ ID NO: 41 - DNA, 50 bp, Synthetic
attgacaagagtgtgttcccctcagtctcaggacggtaaggctgtgcgtt

SEQ ID NO: 42 - DNA, 50 bp, Synthetic
tgcgactcggaggaagaggaggaaggtagcgactgagtgtcttccttccc

SEQ ID NO: 43 - DNA, 50 bp, Synthetic
atgctcttttacccttttcttgcagagcaagaggatttgtataagaaagt

SEQ ID NO: 44 - DNA, 50 bp, Synthetic
catggagttcttggcttcgagaaaagtaagagtcatgcttttcctattca

SEQ ID NO: 45 - DNA, 50 bp, Synthetic
aggatctttctttttcttctacaagtgtatccaccgtgatctggctgcac

SEQ ID NO: 46 - DNA, 50 bp, Synthetic
cccagactatgtccgcaaaggagacgtaagtgcggtagattatccaatta

SEQ ID NO: 47 - DNA, 50 bp, Synthetic
atgtgcttatctttttttttaacaggctagacttcccttaaagtggatgg

SEQ ID NO: 48 - DNA, 50 bp, Synthetic
cttatgtgggagatcttctctcttggtaaatggcactgctgtcctaagag

SEQ ID NO: 49 - DNA, 50 bp, Synthetic
ccattattctctttcttctccccaggtgcctccccttaccctggcttaca

SEQ ID NO: 50 - DNA, 50 bp, Synthetic
ctcctgagtactcctcctctgaaatgtaagaatttgcactgcttgtgtca

SEQ ID NO: 51 - DNA, 50 bp, Synthetic
ttaaacgctttgtgtctcctatgagggaaagcactacatcccgatcaaca

SEQ ID NO: 52 - DNA, 50 bp, Synthetic
ctctcagagactctgggacggcttggtaagatagattcgtctgtttaaca

SEQ ID NO: 53 - DNA, 50 bp, Synthetic
gatctggagtatttgatgctttcaggaacatcaagatccgcccagagagt

SEQ ID NO: 54 - DNA, 50 bp, Synthetic
gaacggaaccaacaagatccacgaggtgaagagcttgtttttctccctca

SEQ ID NO: 55 - DNA, 50 bp, Synthetic
ttaagtgatctttgattgttgacagggtgggcagtcagacagtgggatag

SEQ ID NO: 56 - DNA, 50 bp, Synthetic
gaccccggagtttcatgtcccgagcgtaagtacagtacattcatttccgt

SEQ ID NO: 57 - DNA, 50 bp, Synthetic
aaaaacctgttttcttctccctcaggatgaagaggaaaagtaaggagtca